(19) United States
(12) Patent Application Publication
(10) Pub. No.: US 2018/0346858 A1
(43) Pub. Date: Dec. 6, 2018

(54) ALTERNATE OPERATION CONTROL TYPE MEMBRANE-COUPLED ORGANIC WASTE TREATMENT APPARATUS AND METHOD OF OPERATING THE SAME

(71) Applicant: HYUNDAI ENGINEERING & CONSTRUCTION CO., LTD., Seoul (KR)

(72) Inventors: Young-O Kim, Gyeonggi-do (KR); Ha-young Jang, Gyeonggi-do (KR); Han-saem Lee, Incheon (KR)

(73) Assignee: HYUNDAI ENGINEERING & CONSTRUCTION CO., LTD., Seoul (KR)

(21) Appl. No.: 15/915,167

(22) Filed: Mar. 8, 2018

(30) Foreign Application Priority Data
May 30, 2017 (KR) 10-2017-0066962

(51) Int. Cl. C12M 1/107; C12M 1/00; C12M 1/02; C12P 7/40; C12P 5/02

(52) U.S. Cl. CPC C12M 21/04; C12M 29/04; C12P 5/023; C12P 7/40; C12M 27/00

(57) ABSTRACT

An organic waste treatment apparatus according to the present disclosure includes an acid fermenter 140, a methane fermenter 150, a thickener tank 300, and a separation membrane device 400, and includes: a first circulation line 141 in which a first circulation pump 141a is installed in a linked manner so that a part of organic waste being acid-fermented in the acid fermenter 140 is supplied to the methane fermenter 150; and a second circulation line 151 in which a second circulation pump 151a is installed in a linked manner so that a part of a anaerobic digestive fluid being methane-fermented in the methane fermenter 150 is supplied to the acid fermenter 140, in which the thickener tank 300 is installed between the methane fermenter 150 and the separation membrane device 400, supplied with concentrated circulating water from the separation membrane device 400, and supplies at least a part of the supplied concentrated circulating water to the methane fermenter 150 in an indirect injection manner, such that a means for perfectly removing offensive odor generated during a process of treating the anaerobic digestive fluid is organically coupled, and a high degree treatment process is performed, and as a result, it is possible to perfectly remove offensive odor and to reduce manpower and maintenance costs by automation of the entire facilities.

### CROSS-REFERENCE TO RELATED APPLICATIONS

This application claims the priority of Korean Patent Application No. 10-2017-0066962 filed on May 30, 2017, in the Korean Intellectual Property Office, the disclosure of which is incorporated herein by reference.

### BACKGROUND

### Field

The present disclosure relates to an apparatus and a method for recovering energy from high concentration organic waste and complexly treating an anaerobic digestive fluid, and more particularly, to an alternate operation control type membrane-coupled organic waste treatment apparatus having a new concept and a method of operating the same, in which high concentration organic waste is decomposed by an anaerobic digester, a digestive fluid, in a methane fermenter, of which the organic waste is decomposed, flows into a thickener tank and is then supplied to continuously pass through a separation membrane device in a bidirectional alternate manner, a final concentrated liquid is supplied from a thickener tank to the anaerobic reactor after the digestive fluid is concentrated at high concentration while being repeatedly concentrated and circulated through the thickener tank and the separation membrane device, and the concentrated liquid, which is supplied from the thickener tank to the anaerobic reactor, is mixed with the digestive fluid, vertically and horizontally agitated in the anaerobic reactor, and subjected to a high degree treatment and a gasifying process for high concentration organic waste.

### DESCRIPTION OF THE RELATED ART

In general, the amount of domestic waste is rapidly increased due to industrial development, an increase in income, a change in consumption propensity, and development on distribution structures.

As a selectable solution for treating the domestic waste, there are a method of ensuring a landfill and burying the domestic waste, an incineration method of incinerating the domestic waste, a method of reprocessing the domestic waste to utilize the domestic waste as a new resource, and the like.

The method of burying the domestic waste causes geological pollution, deleterious gas, water pollution, and the like in the landfill after treatment, and involves economic burdens in ensuring a landfill.

In addition, the incineration method involves economic burden in incinerating the waste, causes air pollution during the incineration, and has a limitation in terms of the type of waste to be treated.

In addition, the recycling method appears as an optimum treatment method, but recyclability is determined in accordance with usefulness obtained by recycling the waste. That is, whether to treat the waste is determined based on economic efficiency regarding costs for recycling, secondary environmental pollution that is produced during the recycling process, quality satisfaction with a final product obtained by recycling, and the like.

In the related art, high concentration organic waste (food waste, sewage sludge, waste water sludge, livestock waste, etc.) produced as waste in a general dietary life is treated by using the burying method. However, in the case in which the high concentration organic waste is buried in a landfill, water pollution and offensive odor caused by decomposition are generated, which causes severe environmental pollution. In addition, there occur various types of uneconomic, unhygienic, and non-environmental problems such as a change in soil properties after burying the waste, offensive odor caused by gas generated by decomposition of organic substances, air pollution, and water pollution. Because of these problems, it is forbidden to bury high concentration organic waste in many countries as well as Korea.

Moreover, recently, due to problems of greenhouse and problems with energy, a method of producing bioenergy by using high concentration organic waste is favored all over the world. That is, the high concentration organic waste is considered as an energy source capable of producing new renewable energy instead of an object to be treated. Therefore, there is proposed a method of producing methane gas from organic waste through an anaerobic digestive process and discharging a digestive fluid after purifying the digestive fluid, and to this end, an organic waste treatment apparatus has been developed.

Therefore, there is proposed a recycling method of removing other toxic gas and sludge while extracting methane gas from organic waste, and releasing the digestive fluid after purifying the digestive fluid, and to this end, an organic waste treatment apparatus has been developed.

The organic waste treatment apparatus in the related art will be briefly described. Methane gas, which is produced when the organic waste reacts with microorganisms in an anaerobic reactor of an acid fermenter and a methane fermenter, is separately stored, and a digestive fluid discharged from the methane fermenter is utilized as compost, or purified through a biological treatment process and a physicochemical treatment process.

Recently, there is proposed a membrane-coupled anaerobic digestion technology in which a solid-liquid separation membrane is coupled to an anaerobic digester in order to improve anaerobic digestion efficiency and simplify a digestive fluid treatment process. This technology solid-liquid separates the digestive fluid discharged from the methane fermenter by using the separation membrane device, and transports the separated and solidified material to the anaerobic digester.

Here, agitators including agitation blades are installed in the acid fermenter and the methane fermenter in order to improve reactivity between the organic waste and the microorganisms.

In addition, the concentrated liquid filtered by the separation membrane device is transported directly to the methane fermenter and the acid fermenter, thereby maintaining concentration of the microorganisms in the methane fermenter and the acid fermenter, and increasing treatment efficiency by increasing contact time between the microorganisms and the biodegradable solidified material that is slow in decomposition.

In addition, the digestive fluid to be supplied to the separation membrane device flows only in one direction and is solid-liquid separated.

However, in the organic waste treatment apparatus in the related art which is configured as described above, the agitation caused by the agitator and the reaction between the organic waste and the microorganisms occur actively only at the periphery of a place where the agitation blades are disposed, but the agitation efficiency is significantly decreased as a distance from the agitation blade is increased. In addition, a concentrated liquid, which is concentrated once in the filtration separation membrane device, is transported to the anaerobic reactor, but according to a result of observing the process after a predetermined period, fermentation and decomposition efficiency in the anaerobic reactor rapidly deteriorate, and for this reason, there occurs a problem with a temperature loss of a methane fermented liquid, an impact load caused by a rapid concentration operation, and a large amount of circulating fluid to be filtered. In addition, because the digestive fluid passes through the filtration separation membrane device in one direction, accumulation of contaminants is rapidly increased, and as a result, there is a problem in that a supply flow rate is decreased and it is difficult to operate the separation membrane device.

### DOCUMENT OF RELATED ART

(Patent Document 0001) Korean Patent Application No. 10-2015-0123061

### SUMMARY

The present disclosure has been made in an effort to solve the aforementioned problems, and a first object is to provide an alternate operation control type membrane-coupled organic waste treatment apparatus and a control method, which improve agitation performance for completely mixing high concentration organic waste and maximize a reaction between the organic waste and microorganisms by adding vertical agitation made by forming vortexes in addition to horizontal agitation made by an agitator.

In addition, a second object of the present disclosure is to provide an alternate type membrane-coupled organic waste treatment apparatus and a control method, which allow a digestive fluid to be concentrated to a high degree while repeatedly circulating through a filtration separation membrane device and a thickener tank and then to be supplied back to an anaerobic reactor, and ensure smooth discharge of the digestive fluid or a concentrated liquid by compensating for negative pressure in the thickener tank.

In addition, a third object of the present disclosure is to provide an alternate type membrane-coupled organic waste treatment apparatus and a control method, which control an operation in a bidirectional alternate manner for changing, every predetermined time, an inputting direction in which a digestive fluid flows into a filtration separation membrane device, thereby significantly reducing accumulation of contaminants such as sludge cakes in the filtration separation membrane device, always maintaining efficiency within a constant range, and extending a lifespan of a separation membrane.

Technical problems to be solved by the present disclosure are not limited to the aforementioned technical problem, and other technical problems, which are not mentioned above, may be clearly understood from the following descriptions by those skilled in the art to which the present disclosure pertains.

To achieve the aforementioned objects, an apparatus for recovering energy from high concentration organic waste according to the present disclosure includes an acid fermenter 140, a methane fermenter 150, a thickener tank 300, and a filtration separation membrane device 400, and includes: a first supply line 141 in which a first supply pump 141a is installed in a linked manner so that a part of the organic waste being acid-fermented in the acid fermenter 140 is supplied to the methane fermenter 150 through a heat exchanger 160; a first circulation line 143 in which a first circulation pump 143a is designed in a linked manner so that a part of the organic waste being acid-fermented in the acid fermenter 140 is circulated via the heat exchanger 160; an agitator 154 which is installed in the methane fermenter 150 to smoothly mix an anaerobic digestive fluid being methane-fermented; the agitator 154 which is mounted with an inclined blade 154c for decomposing scum at an upper side of the methane fermenter 150, vertically mixing the anaerobic digestive fluid, and inducing formation of a turbulent flow in the methane fermenter 150; and a second circulation line 230 in which a second circulation pump 231 is installed in a linked manner so that a part of the anaerobic digestive fluid being methane-fermented in the methane fermenter 150 is circulated to the methane fermenter 150 via the heat exchanger 160; in which an inclined plate 260 is installed in the methane fermenter 150 to induce, together with the agitator 154, formation of a turbulent flow of the anaerobic digestive fluid being methane-fermented and induce deposition and discharge of concomitants, and the thickener tank 300 is installed between the methane fermenter 150 and the separation membrane device 400, supplied with concentrated circulating water from the separation membrane device 400, and supplies at least a part of the supplied concentrated circulating water to the methane fermenter 150.

In addition, the separation membrane device 400 is formed in the form of a membrane having a pipe shape, and the membrane having the pipe shape may be disposed to be twisted in at least one of horizontal and vertical directions.

In addition, the fluid flowing into the separation membrane device 400 is moved in a first direction for a preset first period to supply the concentrated circulating water to the thickener tank 300, and when the first period is passed, the fluid may be moved in a second direction opposite to the first direction to supply the concentrated circulating water to the thickener tank 300.

In addition, the separation membrane device 400 may periodically remove organic fouling and inorganic fouling by using a fluid including at least one of water, citric acid, NaOH, and NaOCl.

Meanwhile, to achieve the aforementioned objects, a method of treating organic waste according to the present disclosure includes: a first step S10 of supplying organic waste in an acid fermenter 110 and a methane fermenter 150 and fermenting the organic waste; a second step S20 of storing methane gas generated in the first step S10 in a gas storage tank 140; a third step S30 of introducing and discharging a digestive fluid discharged from the methane fermenter 120 or a concentrated liquid discharged from the a separation membrane device 400 to/from a thickener tank 300 through a supply line 301; a fourth step S40 of injecting, by a supply pump 320, the digestive fluid stored in the thickener tank 200 into the separation membrane device 400 to separate the digestive fluid into a concentrated liquid and filtered water, and reversing an injection direction of the digestive fluid every predetermined time; a sixth step S60 of transporting the separated concentrated liquid to the acid fermenter 140 and the methane fermenter 150; a seventh step of allowing the thickener tank 300 to be supplied with concentrated circulating water from the separation membrane device 400; and an eighth step of supplying at least a part of the concentrated circulating water supplied to the thickener tank 300 to the methane fermenter 150.

In addition, the fluid flowing into the separation membrane device 400 is moved in a first direction for a preset first period to supply the concentrated circulating water to the thickener tank 300, and when the first period is passed, the fluid may be moved in a second direction opposite to the first direction to supply the concentrated circulating water to the thickener tank 300.

In addition, the method may further include an eighth step of periodically removing organic fouling and inorganic fouling by using a fluid including at least one of water, citric acid, NaOH, and NaOCl in the separation membrane device 400.

According to the present disclosure, a turbulent flow is formed at both of the upper and lower sides of the methane fermenter 150 by vertical and horizontal dual forced agitation, such that agitation performance of the organic waste is maximized, as a result, it is possible to improve efficiency in contact with the microorganisms.

In addition, the organic waste at the upper sides of the acid fermenter and the methane fermenter is circulated to the lower sides of the acid fermenter and the methane fermenter, such that the organic waste is not deposited, and oil of the organic waste at the upper side is efficiently mixed and agitated to improve contact with the microorganism.

In addition, pH is adjusted as some organic substances, which are being fermented in the acid fermenter and the methane fermenter, are circulated, pH becomes uniform by dual agitation in the acid fermenter and the methane fermenter, and as a result, fermentation efficiency is maximized.

In addition, indirect injection is applied in which the digestive fluid discharged from the methane fermenter is concentrated to a high degree while repeatedly circulating through the thickener tank and the separation membrane device and supplied to an anaerobic reactor, and as a result, a state of the anaerobic microorganisms in an anaerobic reaction tank is stabilized. In addition, with the inflow of the concentrated liquid with high concentration, concentration of the anaerobic microorganisms and the solid retention time (SRT) are maintain at a high level without a loss of anaerobic microorganisms which are slow in growth, and for this reason, the decomposition of the organic substance becomes active, such that the amount of generated gas is also increased.

In addition, in comparison with a reduction in microorganism activity and microorganism floc in accordance with daily circulation amount performed in the methane fermenter, a process of recirculation to the thickener tank and the separation membrane device is repeated, and then the concentrated liquid with high concentration flows into the methane fermenter, thereby improve an ecological environment for microorganisms.

In addition, the negative pressure generated in the thickener tank when the digestive fluid or the concentrated liquid is discharged from the thickener tank is compensated by the gas from the digestive fluid, thereby smoothly discharging the digestive fluid or the concentrated liquid.

In addition, the circulation direction of the digestive fluid or the concentrated liquid to be supplied to the separation membrane device is periodically changed, and as a result, it is possible to significantly prevent membrane contamination caused by accumulation of contaminants on the tubular filtration membrane, thereby remarkably improving a lifespan of the filtration membrane. For this reason, it is possible to minimize cost incurred due to frequent replacement of the expensive filtration membrane and to prevent deterioration in efficiency of the separation membrane device.

In addition, the separation membrane device is efficiently cleaned by water cleaning or chemical cleaning, and as a result, it is possible to further increase a lifespan of the filtration membrane.

Therefore, in the present disclosure, the thickener tank 300 is disposed between the separation membrane device 400 and the methane fermenter 150, such that the existing circulation method by 90 Q (quarter) is reduced to 30 to 35 Q, and as a result, it is possible to prevent the floc (microorganism community) from being broken, and in turn, it is possible to generate methane gas by 20% or more and to increase efficiency in decomposing the organic substance by 90% or more.

In addition, the methane fermenter 150, the thickener tank 300, and the separation membrane device 400 are installed in a hermetic closed circulation manner, and the anaerobic digestive sludge contained in the concentrated circulating water is transported to the methane fermenter 150 once more, such that it is possible to induce an effect of facilitating the decomposition of the organic substance in the methane fermenter 150, it is possible to basically block offensive odor generated during the wastewater treatment for the anaerobic digestive fluid, and it is possible to reduce, by 30% or more, the amount of generated sludge to be wasted by the recirculation of the concentrated liquid with high concentration.

In addition, there may be a problem when only horizontal agitation is performed in the methane fermenter 150 and vertical agitation is partially performed, but in the present disclosure, it is possible to enable forced horizontal and vertical agitation effects by using indirect injection to the methane fermenter 150 with concentration circulation in the thickener tank 300 and the separation membrane device 400. That is, it is possible to improve a dual mixing effect.

In addition, the separation membrane device 400 according to the present disclosure is operated together with the thickener tank 300 in an alternate operation control manner, and moves a fluid in a first direction for a predetermined period, and the separation membrane device 400 moves the fluid in a second direction opposite to the first direction for a next period, such that with the swing operation method, the sludge is not deposited and may continuously flow and move in the membrane pipe.

In addition, in the present disclosure, water is inputted first for cleaning and then discharged, and water and NaOCl are inputted for cleaning and then discharged in order to remove organic fouling such as deposition of the sludge, and finally, only in a case in which the amount of flux (permeable water) is significantly decreased, it is possible to remove inorganic fouling by performing washing while inputting water and NaOH or water and citric acid.

Meanwhile, the effects obtained by the present disclosure are not limited to the aforementioned effects, and other effects, which are not mentioned above, will be clearly understood by those skilled in the art from the following description.

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a configuration view schematically illustrating a membrane-coupled apparatus for recovering energy from organic waste having an alternation concept according to an exemplary embodiment of the present disclosure;

FIG. 2 is a state view schematically illustrating an acid fermenter and a methane fermenter in FIG. 1;

FIG. 3 is a view schematically illustrating an interior of the methane fermenter illustrated in FIG. 2;

FIGS. 4 and 5 are cross-sectional side views schematically illustrating a state in which a gas holder installed in a thickener tank illustrated in FIG. 1 is operated;

FIG. 6 is a schematic cross-sectional view of a membrane filtration module according to the present disclosure;

FIG. 7 is a view illustrating an example of a structure for transporting concentrated sludge in concentrated circulating water to the methane fermenter 150 by using the thickener tank in accordance with the present disclosure;

FIG. 8 is a view illustrating a specific example of a structure for transporting the concentrated circulating water from a separation membrane device to the thickener tank in accordance with the present disclosure;

FIGS. 9A & 9B are views illustrating a specific example of an internal structure of the separation membrane device described with reference to FIG. 8;

FIGS. 10A & 10B provided views for explaining a method of removing organic fouling and inorganic fouling that may be applied to the present disclosure; and

FIG. 11 is a block diagram illustrating an organic waste treatment and operation method using an alternate operation control type membrane-coupled organic waste treatment apparatus.

### DETAILED DESCRIPTION OF THE PREFERRED EMBODIMENT

Hereinafter, an alternate operation control type membrane-coupled organic waste treatment apparatus according to an exemplary embodiment of the present disclosure will be described in detail with reference to the accompanying drawings.

<Configuration>

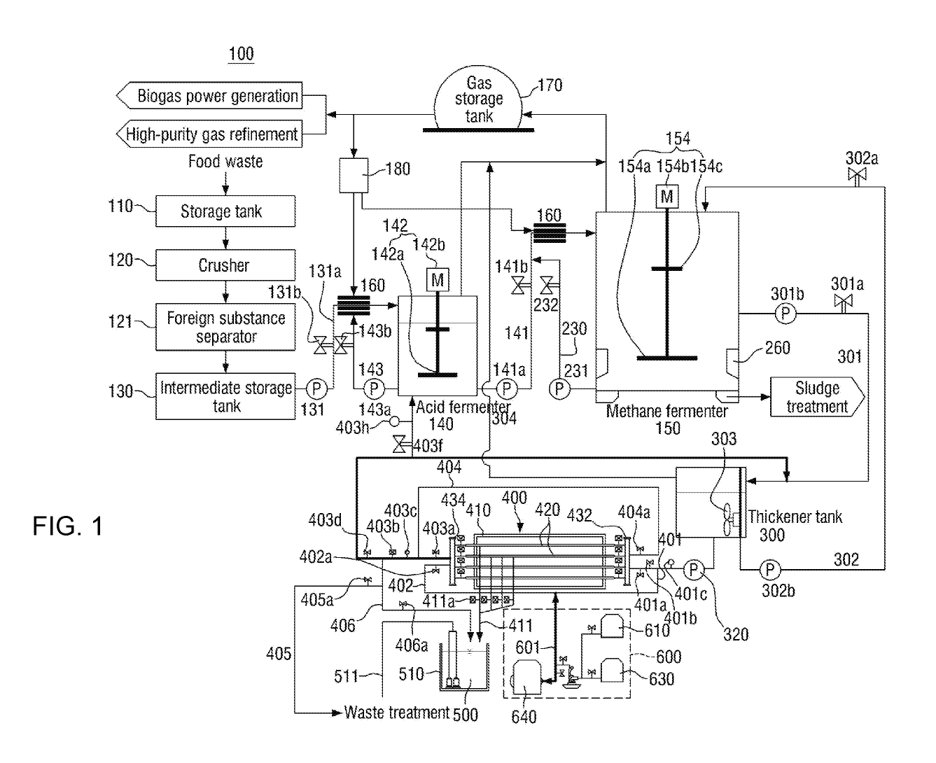

First, as illustrated in FIG. 1, an organic waste treatment apparatus 100 according to an exemplary embodiment of the present disclosure includes a storage tank 110, a pulverizer 120, a foreign substance separator 121, an intermediate storage tank 130, an acid fermenter 140, a methane fermenter 150, heat exchangers 160 which are installed in the acid fermenter 140 and the methane fermenter 150, respectively, a gas storage tank 170, a first digestive fluid inflow line 301, a thickener tank 300, a supply pump 320, a separation membrane device 400, a first concentrated liquid inflow line 401, an injection direction changing means, a first concentrated liquid transport line 403, a linked storage tank 500, and a cleaning means 600.

Here, the storage tank 110 stores organic waste for about 2 to 3 days, the crusher 120 is a device for crushing and cutting medium-sized or large-sized solidified materials of the organic waste to a size of 15 to 25 mm in order to facilitate smooth foreign substance separation, and the foreign substance separator 121 is a device for separating foreign substances such as synthetic resin or metal contained in the organic waste which are not biologically decomposed.

The crusher 120 is a device for pulverizing the crushed organic waste into small pieces, and the organic waste pulverized by the crusher 120 contains moisture of about 80 to 85%, such that fluidity is comparatively high.

The foreign substance separator 121 separates vinyls, solidified materials of 5 mm or larger, and inert materials which are contained in the organic waste crushed into small pieces, thereby further increasing fluidity.

The intermediate storage tank 130 temporarily stores the organic waste discharged from the crusher 120, and the intermediate storage tank 130 is provided with a conveying pump 131 for conveying the organic waste in the intermediate storage tank 130 to the acid fermenter 140.

The heat exchangers 160 are connected to a boiler 180 and heat the organic waste to a temperature of approximately 65 to 70° C., thereby maintaining the organic waste in the acid fermenter 140 and the methane fermenter 150 at a temperature of approximately 35 to 55° C. The reason is to maximize growth of anaerobic microorganisms and fermentation of the organic waste with a high oil content.

The gas storage tank 170 stores biogas produced in the acid fermenter 140 and the methane fermenter 150, a part of the biogas stored in the gas storage tank 170 is supplied to the boiler 180, and another part of the biogas is sent to a high-purity gas refinery or supplied to a biogas power generation system so as to be used to generate electric power.

The boiler 180 supplies heat to the heat exchanger 160 by using the biogas stored in the gas storage tank 170 as an energy source.

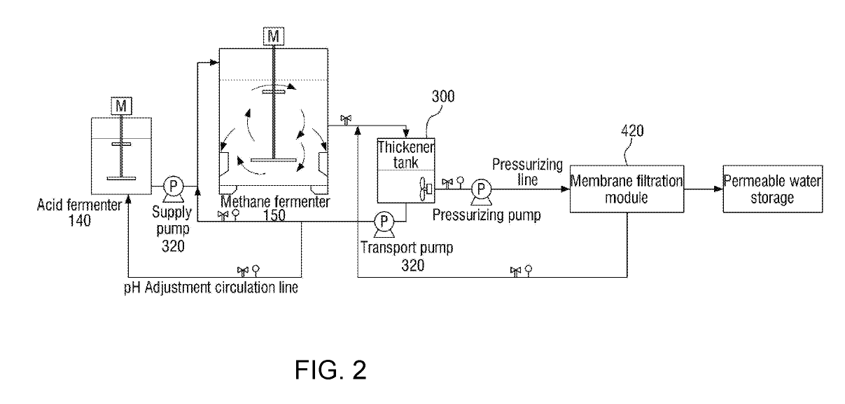

Meanwhile, as illustrated in FIG. 2, the acid fermenter 140 includes a first supply line 141 for supplying the acid-fermented organic substance to the methane fermenter 150, a first agitator 142 installed in the acid fermenter 140, and an agitation circulation line 143 for circulating the organic waste at an upper side to a lower side.

Here, the first agitator 142 is installed such that a first agitation blade 142a positioned in the acid fermenter 140 is rotated by a first agitation motor 142b. Here, the first agitation motor 142b may operate at 15 to 25 RPM, an internal pressure of the first agitation motor 142b will be dangerously increased if the rotational speed is above 25 RPM, and agitation performance will deteriorate if the rotational speed is below 15 RPM.

In addition, an agitation circulation pump 143a, which is connected between an upper side and a lower side of the acid fermenter 140 and circulates the organic waste at the upper side of the acid fermenter 140 to the lower side of the acid fermenter 140, is installed in the agitation circulation line 143 in a linked manner. The first agitation circulation pump 143a may have a capacity capable of circulating the organic waste at a flow rate equal to or lower than 6 to 8 times an inflow rate of the organic waste flowing into the acid fermenter 140 in order to prevent an anaerobic microorganism floc from being broken.

Here, the first supply line 141 may be installed to supply the acid-fermented organic substance from the lower side of the acid fermenter 140 to the upper side of the methane fermenter 150, and a predetermined amount of organic waste may be circulated for approximately every 1 to 3 hours.

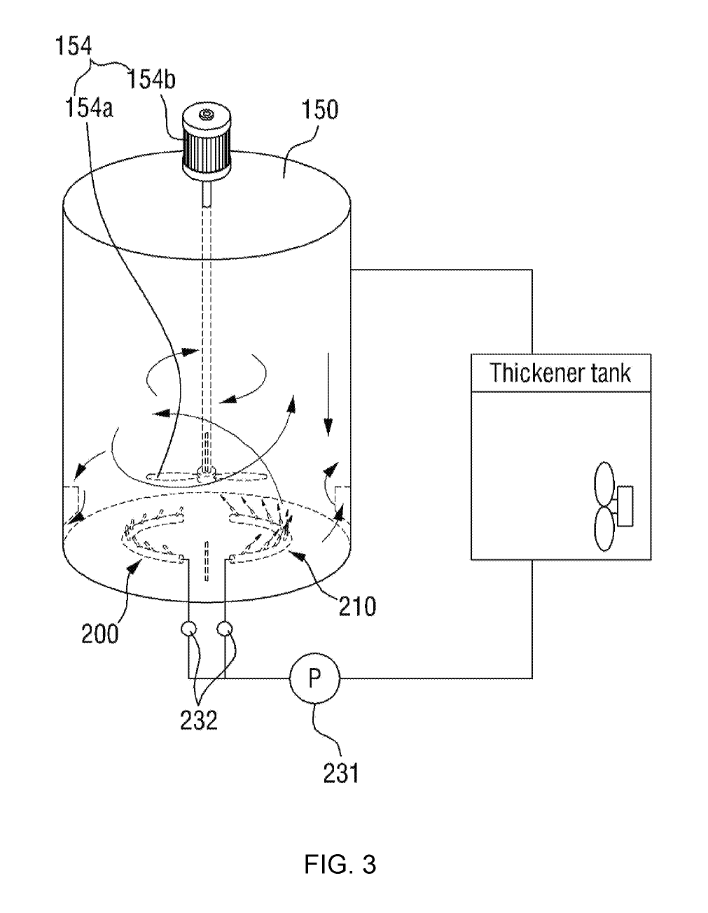

Meanwhile, as illustrated in FIGS. 2 to 3, the methane fermenter 150 includes a second agitator 154 and inclined plates 260 which are installed so that an anaerobic digestive fluid is mixed and fermented by forming a turbulent flow, and a second circulation line 230 which circulates a part of the fermented anaerobic digestive fluid in the methane fermenter 150. In addition, the second circulation line 230 may have a capacity capable of circulating the organic waste at a flow rate of 2 to 4 times an inflow rate of the organic waste flowing from the acid fermenter 140.

Here, the second agitator 154 is installed such that second agitation blades 154a and 154c positioned in the methane fermenter 150 are rotated by a second agitation motor 154b. The second agitation motor 154b may operate at 15 to 25 RPM, an internal pressure of the second agitation motor 154b will be dangerously increased if the rotational speed is above 25 RPM, and agitation performance will deteriorate if the rotational speed is below 15 RPM.

Here, the second agitation blade 154a is installed to induce horizontal agitation in the methane fermenter 150, the second agitation blade 154c is installed to induce upper scum crushing and vertical agitation by adjusting an angle of the agitation blade, thereby allowing biogas generated in the methane fermenter 150 to be smoothly discharged.

Here, the methane fermenter 150 includes the plurality of inclined plates 260 formed at a rim portion of a bottom surface of the methane fermenter 150, a sludge groove 152, and a sludge discharge line 153 which is connected to the sludge groove 152 and installed to discharge sludge, and a bottom surface of the methane fermenter 150 is inclined from a center to the rim portion.

The sludge groove 152 may be formed below a position at which the inclined plate 260 is formed, and the reason is to enable the sludge moving downward by the inclined plate 260 to be collected directly to the sludge groove 152.

In addition, the inclined bottom surface of the methane fermenter 150 serves to allow the sludge flowing on the bottom surface to flow to the rim portion of the bottom surface and then to be collected in the sludge groove 152.

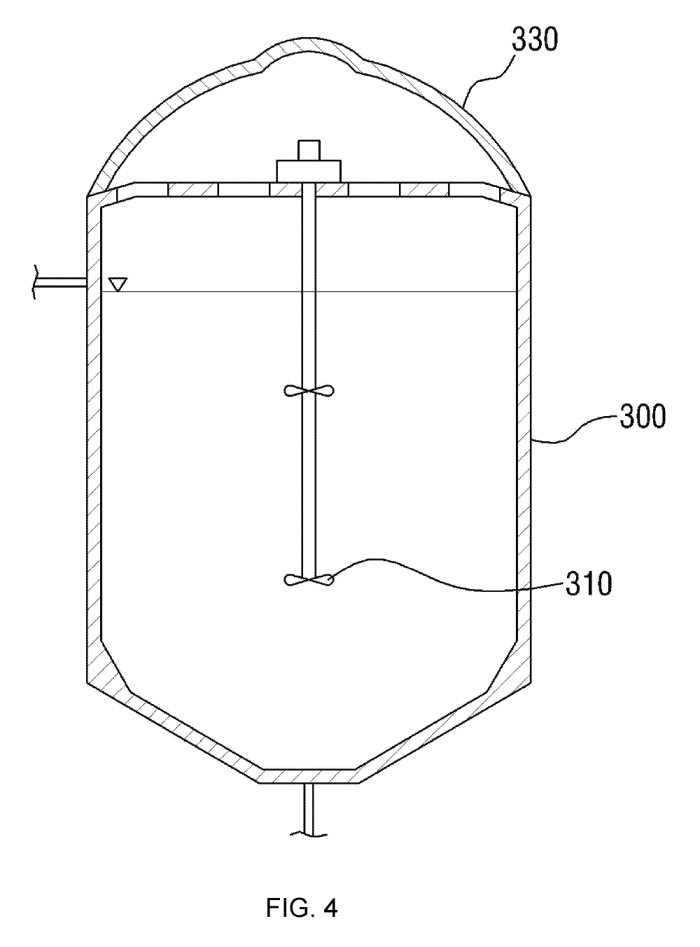

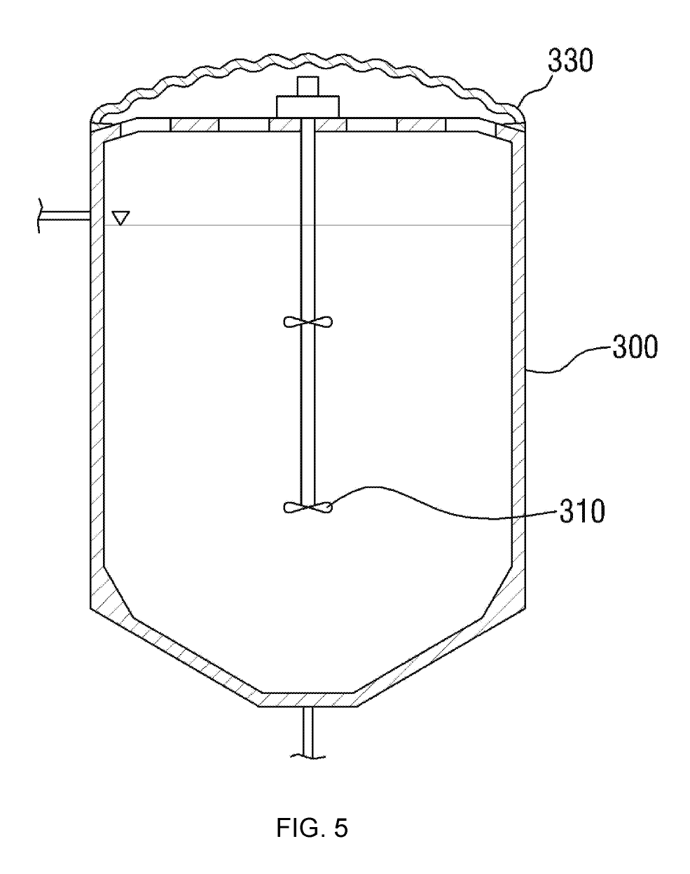

Meanwhile, the thickener tank 300 is installed such that the digestive fluid discharged from the methane fermenter 150 flows into the thickener tank 300 through a supply line 301, and a third agitator 303, which agitates the digestive fluid so that the digestive fluid does not settles when the digestive fluid flows in, is embedded in the thickener tank 300. In addition, as illustrated in FIGS. 4 and 5, a first concentrated liquid transport line 403 to be described below is connected to the supply line 301 of the thickener tank 300. In addition, a function of the thickener tank 300 for producing a concentrated liquid with high concentration will also be described in the description of the separation membrane device 400.

In addition, a first concentrated liquid circulation line 403h circulates a part of an anaerobic digestive fluid concentrate of the separation membrane device 400 to the acid fermenter 140. The reason is to reduce acidity in the acid fermenter 140 in order to avoid a phenomenon in which the acid-fermented organic substance, which is agitated in the acid fermenter 140, has strong acidity of about pH 3 to 4 and the fermentation is not smoothly performed in the acid fermenter 140.

A gas holder 330 is made of a soft material and installed to be expandable and contractible.

Meanwhile, a first digestive fluid inflow line 401 connects the thickener tank 300 and the separation membrane device 400 and serves as a movement passage for the digestive fluid. Further, a first flowmeter 401b for measuring an injection flow rate of the digestive fluid is provided in the first digestive fluid inflow line 401. Further, a first manometer 401c for measuring an injection pressure of the digestive fluid may be provided separately from the first flowmeter 401b.

The supply pump 320 is provided in the first digestive fluid inflow line 401 and serves to pressurize and convey the digestive fluid stored in the thickener tank 300 to the separation membrane device 400. The supply pump 320 may operate in conjunction with a level adjusting device (not illustrated) of the thickener tank 300, first, second, and third flowmeters 401b, 403b, and 411a, or first and second manometers 401c and 403c, and the supply pump 320 may be inverter-controlled by a control unit (not illustrated) so that a pumping speed thereof may be adjusted.

The separation membrane device 400 serves to solid-liquid separate the digestive fluid conveyed by the supply pump 320 into a concentrated liquid and filtered water. According to the present exemplary embodiment, as illustrated in FIG. 1, the separation membrane device 400 includes a frame 410, membrane filtration modules 420, and first and second headers 432 and 434.

Here, the frame 410 serves to accommodate and support the membrane filtration modules 420.

The first and second header 432 and 434 are tubular bodies having a predetermined internal space. The first header 432 allows the first digestive fluid inflow line 401 to communicate with the plurality of membrane filtration modules 420, and the second header 434 allows the plurality of membrane filtration modules 420 to communicate with the first concentrated liquid transport line 403. Therefore, the digestive fluid, which flows into the first header 432 through the first digestive fluid inflow line 401, is distributed to the plurality of membrane filtration modules 420, and the digestive fluid, which is discharged from the plurality of membrane filtration modules 420, is collected in the second header 434 and discharged to the first concentrated liquid transport line 403.

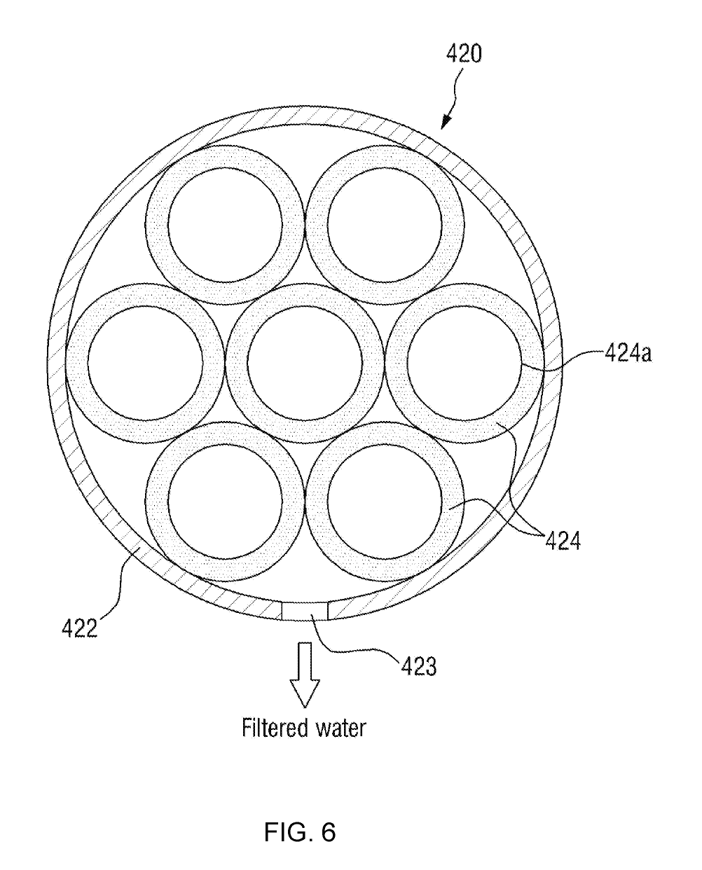

The membrane filtration module 420 has a cylindrical shape having a predetermined length, and an interior of the membrane filtration module 420 is filled with filtration membranes. According to the present exemplary embodiment, one or more membrane filtration modules 420 are provided, and the membrane filtration module 420 is embedded and installed in the frame 410 so that both ends thereof protrude outward from the frame 410. As one exemplary embodiment, as illustrated in FIG. 1, one or more membrane filtration modules 420 are provided, and six modules 420 may be configured as one set at each end thereof (not illustrated). In addition, as illustrated in FIG. 6, in the membrane filtration module 420, a plurality of tubular filtration membranes 424, in the form of a bundle, is embedded in a cylindrical casing 422. Further, the casing 422 has a filtered water discharge hole 423, and the filtered water discharge line 411 is connected to the filtered water discharge hole 423. The symbol ⊚ illustrated in FIG. 6 indicates an injection space and an injection direction of the digestive fluid. When the digestive fluid is injected into the tubular filtration membrane 424 at a predetermined pressure, water contained in the digestive fluid alternately penetrates cylindrical membrane surfaces 424a, thereby performing the filtration. Therefore, the tubular filtration membrane 424 has a structure in which the high-concentration concentrated liquid containing digestive microorganisms is discharged at the opposite side to the injection side, and the filtered water is discharged through the discharge hole 423 formed in the casing 422.

Meanwhile, PVDF, ceramic, or the like is used as a material of the tubular filtration membrane 424, and the tubular filtration membrane 424 has a shape in which an interior of a porous pressure-resistant support tube (not illustrated) is coated with a membrane (not illustrated) having fine pores. The tubular filtration membrane 424 is advantageously suitable for a treatment of raw water with high turbidity because it is possible to ensure a wide flow path through the injected raw water passes. In addition, there are advantages in that in comparison with other types of filtration membranes such as a flat plate type, hollow fibers, or a spiral wound type, occlusion less occurs on the membrane surfaces 424a and backwashing is easily performed when cleaning the filtration membrane.

An ultrafiltration (UF) membrane or a microfiltration (MF) membrane may be used as the tubular filtration membrane 420.

The injection direction changing means serves to change the injection direction of the digestive fluid, which is supplied to the separation membrane device 400, to a reverse direction in a preset period of time. According to the present exemplary embodiment, the injection direction changing means includes a second digestive fluid inflow line 402, a second concentrated liquid transport line 404, first and second injection valves 401a and 402a, first and second discharge valves 403a and 404a, and a control unit (not illustrated).

The second digestive fluid inflow line 402 diverges from one side of the first digestive fluid inflow line 401 and is connected to the second header 434 of the separation membrane device 400. Further, the second concentrated liquid transport line 404 connects the first header 432 of the separation membrane device 400 to one side of the first concentrated liquid transport line 403.

A first injection valve 401a is provided in the first digestive fluid inflow line 401 adjacent to the first header 432, and a first discharge valve 403a is provided in the first concentrated liquid transport line 403 adjacent to the second header 434.

A second injection valve 402a is provided in the second digestive fluid inflow line 402 adjacent to the second header 434, and a second discharge valve 404a is provided in the second concentrated liquid transport line 404 adjacent to the first header 432.

The first concentrated liquid transport line 403 connects the separation membrane device 400 and the acid fermenter 140, the methane fermenter 150 is connected to the thickener tank 300, and the first concentrated liquid transport line 403 serves as a passageway through which the concentrated liquid produced in the separation membrane device 400 is transported to the acid fermenter 140 and the thickener tank 300. Further, a second flowmeter 403b for measuring a discharge flow rate of the concentrated liquid is provided in the first concentrated liquid transport line 403. Further, a second manometer 403c for measuring a discharge pressure of the concentrated liquid may be provided separately from the second flowmeter 403b. In addition, a valve, an electronic flowmeter, and the like for adjusting a supply amount of the concentrated liquid may be further provided. In addition, circulation valves 403d and 403f are provided in the first concentrated liquid transport line 403 in order to allow the concentrated liquid discharged from the separation membrane device 400 to flow to the acid fermenter 140 and the thickener tank 300. The circulation valves 403d and 403f are controlled by a controller so as to have an opening and closing operation opposite to opening and closing operations of a supply valve 403f for the acid fermenter 140 and a circulation valve 403d for the thickener tank 300, and have an opening and closing operation opposite to an opening and closing operation of a shut-off valve 301a which shuts off the supply of the methane fermented liquid. That is, a first supply valve 302a and a second transport valve 302a are controlled to be closed in a case in which the circulation valve 403d is controlled to be opened so that the concentrated liquid, which flows through the first concentrated liquid transport line 403, does not flow into the acid fermenter 140 but flows back into the thickener tank 300 and the separation membrane device 400 through the supply line 301. In addition, the second supply valve 403f is controlled to be opened in a case in which the circulation valve 301a is controlled to be closed so that the concentrated liquid, which flows through the first concentrated liquid transport line 403, does not flow into the supply line 301, but flows into the acid fermenter 140. Otherwise, the shut-off valve 301a may be controlled to be opened so that the digestive fluid, which is discharged from the methane fermenter 150, flows by a water level gauge of the thickener tank 300. Here, an electronic flowmeter and an auxiliary valve are further installed in the first concentrated liquid transport line 403 in order to adjust the amount of the concentrated liquid which flows into the acid fermenter 140 and circulates in the thickener tank 300. In addition, electronic flowmeters may be further installed in the supply line 301 between the methane fermenter 150 and the shut-off valve 301a and between the thickener tank 300 and the separation membrane device 400.

The controller is operated by a program programmed based on the number of times the concentrated liquid discharged from the separation membrane device 400 is filtered. In more detail, the controller is programmed such that a low concentrated liquid discharged from the separation membrane device 400 does not flow directly into the acid fermenter 140 and the methane fermenter 150, but flows back to the separation membrane device 400 through the supply line 301 via the thickener tank 300. In addition, the controller controls the first injection valve 401a, which is installed in the supply line 301, so that the entire amount of cleaning water or low concentrated liquid in the thickener tank 300 flows into the separation membrane device 400 by the supply pump 420. In addition, the controller controls opening and closing operations of the first and second injection valves 401a and 402a and the first and second discharge valves 403a and 404a in addition to electric/electronic devices such as various types of pumps and valves mounted in the acid fermenter 140 and the methane fermenter 150, thereby reversing the injection direction of the digestive fluid or the low concentrated liquid. In this case, a cycle on which the injection direction is reversed by the control unit may be appropriately selected in a range from about 30 minutes to about 60 minutes. That is, a forward injection direction in which the digestive fluid or the low concentrated liquid flows to the first digestive fluid inflow line 401 and the first concentrated liquid transport line 403 in the separation membrane device 400 and a reverse injection direction in which the digestive fluid or the low concentrated liquid flows to the second digestive fluid inflow line 402, the second concentrated liquid transport line 404, and the first concentrated liquid transport line 403 are periodically changed. Here, the concentrated liquid with high concentration (referred to as a high concentrated liquid) refers to a concentrated liquid in which an initial digestive fluid is concentrated to a high degree while repeatedly circulating through the thickener tank 300 and the separation membrane device 400, that is, a concentrated liquid immediately before being supplied to an anaerobic reactor. In addition, a concentrated liquid with low concentration (referred to as a low concentrated liquid) refers to a concentrated liquid which is initially discharged from the separation membrane device 400 and is repeatedly circulating. Of course, the digestive fluid refers to a substance which is discharged from the methane fermenter 150 and initially flows into the thickener tank 300.

The linked storage tank 500 serves to temporarily store filtered water discharged from the separation membrane device 400 and the convey the filtered water to a wastewater treatment facility. As illustrated in FIG. 1, the linked storage tank 500 is connected to the separation membrane device 400 through a filtered water discharge line 411, and the filtered water is conveyed to the wastewater treatment facility through a linked line 511 by operating a linked pump 510. Meanwhile, the third flowmeters 411a for measuring a discharge flow rate of the filtered water discharged from the respective membrane filtration modules 420 are provided in the filtered water discharge line 411.

The cleaning means 600 serves to improve a lifespan by cleaning the separation membrane device 400. In this case, the cleaning means 600 is configured such that the control unit derives a difference in flow rate or a difference in pressure based on a flow rate value or a pressure value measured by the first and second flowmeters 401b and 403b or the first and second manometers 401c and 403c, and the control unit operates the cleaning means 600 in a case in which the derived difference in flow rate or the derived difference in pressure is smaller than a predetermined reference value. As illustrated in FIG. 1, the cleaning means 600 according to the present exemplary embodiment includes a cleaning water supply line 601, a water tank 640, a backwashing pump 620, and chemical tanks 610 and 630, and enables water cleaning or chemical cleaning.

The cleaning water supply line 601 is connected to the second digestive fluid inflow line 402.

The backwashing pump 620 pressurizes and conveys cleaning water stored in the water tank 610 to the separation membrane device 400 through the cleaning water supply line 601 and the second digestive fluid inflow line 402.

The chemical tanks 610 and 630 are connected to the cleaning water supply line 601 and supply a cleaning chemical, thereby enabling the chemical cleaning. NaOCl (sodium hypochlorite), citric acid, NaOH, and the like may be used as the cleaning chemical.

Meanwhile, first and second cleaning solution transport lines 405 and 406 and fifth, sixth, and seventh valves 403d, 405a and 406a are provided to discharge the cleaning solution to the wastewater treatment facility after cleaning the separation membrane device 400.

The fifth valve 403d is provided in the first concentrated liquid transport line 403 and closed when cleaning the separation membrane device 400, thereby allowing the cleaning solution to flow into the first cleaning solution transport line 405 and the second cleaning solution transport line 406.

A first cleaning solution discharge line 405 diverges from the first concentrated liquid transport line 403, and the sixth valve 405a is provided in the first cleaning solution discharge line 405. A second cleaning solution transport line 406 diverges from the first cleaning solution transport line 405 and is connected to the wastewater treatment linked storage tank 500. In this case, the seventh valve 406a is provided in the second cleaning solution transport line 406.

Meanwhile, there separately occurs a problem in that offensive odor is created during a process of treating the organic waste.

The present disclosure proposes a structure for transporting concentrated circulating water from the separation membrane device 400 to the thickener tank 300 in order to solve the problem.

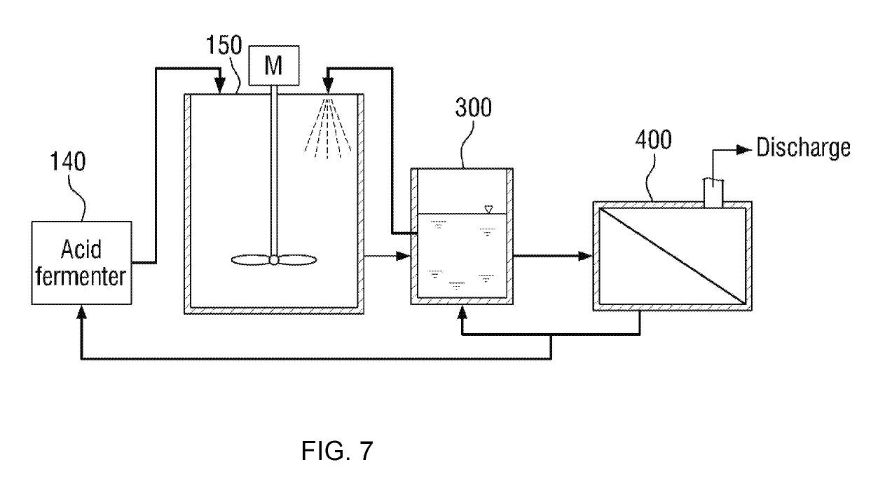

FIG. 7 is a view illustrating an example of a structure for transporting concentrated sludge in concentrated circulating water to the methane fermenter 150 by using the thickener tank 300 in accordance with the present disclosure.

The thickener tank 300 is disposed between the separation membrane device 400 and the methane fermenter 150, but if the thickener tank 300 is not disposed between the separation membrane device 400 and the methane fermenter 150, excessive mixing occurs in the methane fermenter 150, and there is a problem with an automatic operation and control of the separation membrane device 400.

The excessive mixing causes an effect of breaking a floc (microorganism community), the amount of generated methane gas is decreased accordingly, and an effect of decomposing hazardous substances also deteriorates.

Therefore, in the present disclosure, the thickener tank 300 is disposed between the separation membrane device 400 and the methane fermenter 150, such that the existing circulation method by 90 Q (quarter) is reduced to 30 to 35 Q, and as a result, it is possible to prevent the floc (microorganism community) from being broken, and in turn, it is possible to generate methane gas by 20% or more and to increase efficiency in decomposing the organic substance by 90% or more.

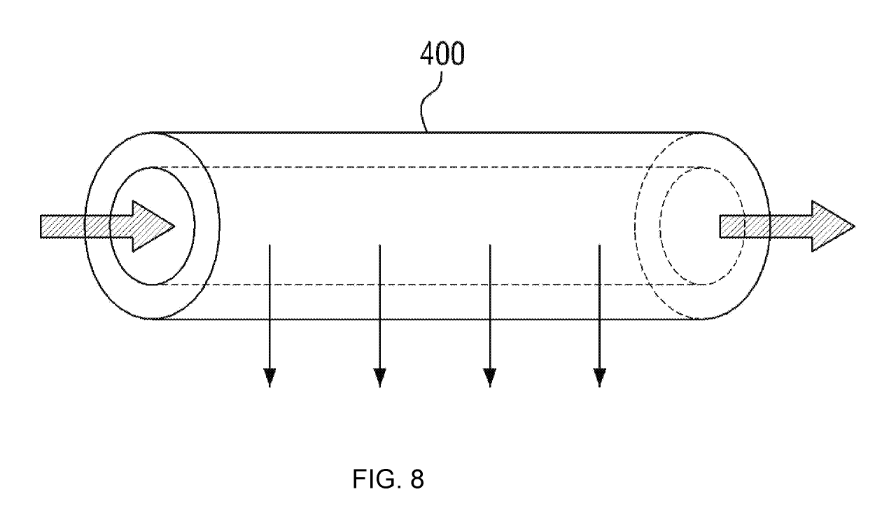

In addition, FIG. 8 is a view illustrating a specific example of a structure for transporting the concentrated circulating water from the separation membrane device to the thickener tank 300 in accordance with the present disclosure.

Referring to FIG. 8, it is possible to prevent offensive odor produced in the existing wastewater treatment facility due to the fluid passing through the separation membrane device 400.

For example, the anaerobic digestive fluid and the concentrated circulating water, which cause offensive odor, contain anaerobic digestive sludge, and when the filtered water is filtered, the concentrated circulating water having the anaerobic digestive sludge with high concentration is recirculated without being discharged to the outside from the thickener tank 300 to the methane fermenter 150, and as a result, it is possible to basically block offensive odor.

Referring to FIG. 7, in the present disclosure, the concentrated circulating water, which contains a large amount of anaerobic digestive sludge, is transported to the thickener tank 300 instead of being discharged to the outside.

In the present disclosure, the anaerobic digestive sludge contained in the concentrated circulating water is transported to the methane fermenter 150 once more, and as a result, it is possible to induce an effect of increasing the amount of microorganisms in the methane fermenter 150 and the solid retention time (SRT) of the microorganisms and facilitating the decomposition of organic substances, and it is possible to prevent offensive odor from being produced.

In the case in which the anaerobic digestive sludge contained in the concentrated circulating water is transported to the methane fermenter 150 once more, it is possible to increase the amount of generated biogas by 20% or more and decrease the amount of sludge to be wasted by 30% or more in comparison with the existing method.

There may be a problem when only horizontal agitation is performed in the methane fermenter 150 and vertical agitation is partially performed, but in the present disclosure, it is possible to enable forced horizontal and vertical agitation effects by using indirect injection to the methane fermenter 150 with concentration circulation in the thickener tank 300 and the separation membrane device 400. That is, it is possible to improve a dual mixing effect.

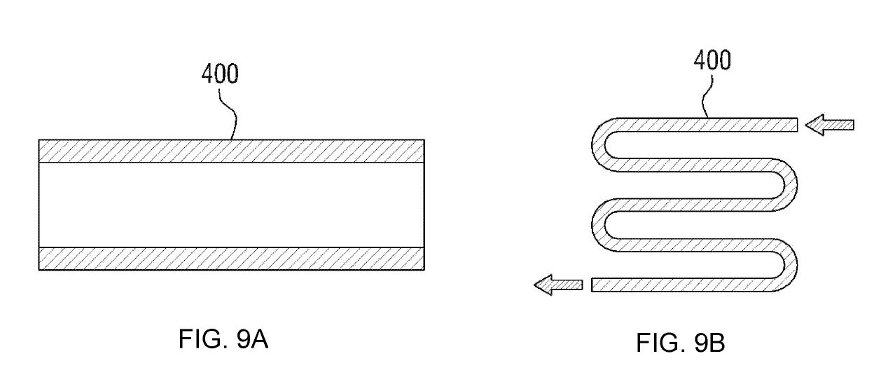

Meanwhile, FIGS. 9A & 9B are views illustrating a specific example of an internal structure of the separation membrane device described with reference to FIG. 8.

Referring to FIGS. 9A & 9B, the separation membrane device 400 according to the present disclosure is configured by a membrane in the form of a pipe.

As illustrated in FIG. 9A, the separation membrane device 400 according to the present disclosure may be formed to have a length of 3 m or more, and an external surface of the separation membrane device 400 may be implemented in the form of a membrane pipe.

In addition, the separation membrane device 400 is implemented in a twisted shape, such that the separation membrane device 400 has a shape in which a membrane pipe is entangled and twisted in a vertical or horizontal direction when viewed from the front side as illustrated in FIG. 9B.

In a case in which the separation membrane device 400 according to the present disclosure is operated together with the thickener tank 300 in a swing manner and moves the fluid in a first direction for a predetermined period, the separation membrane device 400 moves the fluid in a second direction opposite to the first direction for a next period.

With this swing operation method, the sludge is not deposited and may continuously flow and move in the membrane pipe.

Even in the case in which the swing operation method is used, there may be a problem in that the sludge is deposited on an inner wall of a pipe.

Therefore, in the present disclosure, it is possible to solve the problem of deterioration in penetration amount and an increase in pressure caused by periodic organic fouling and periodic inorganic fouling.

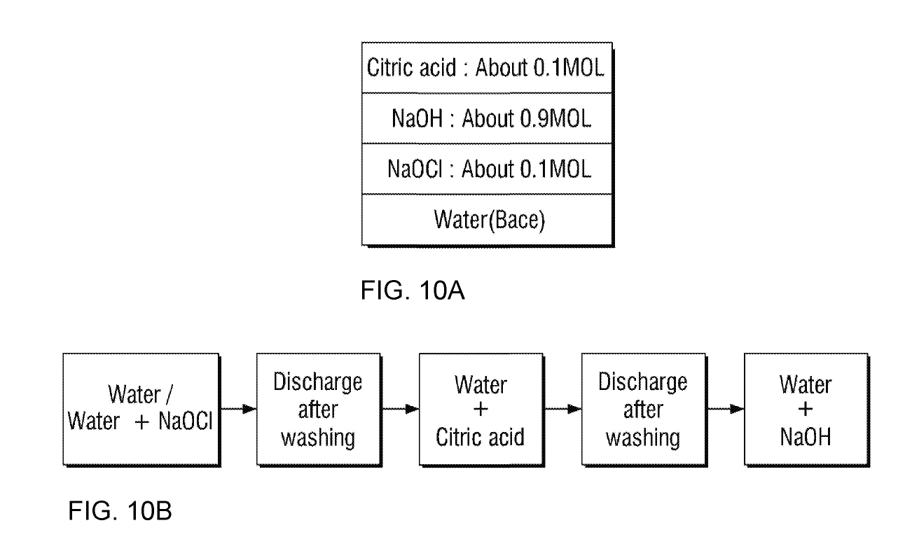

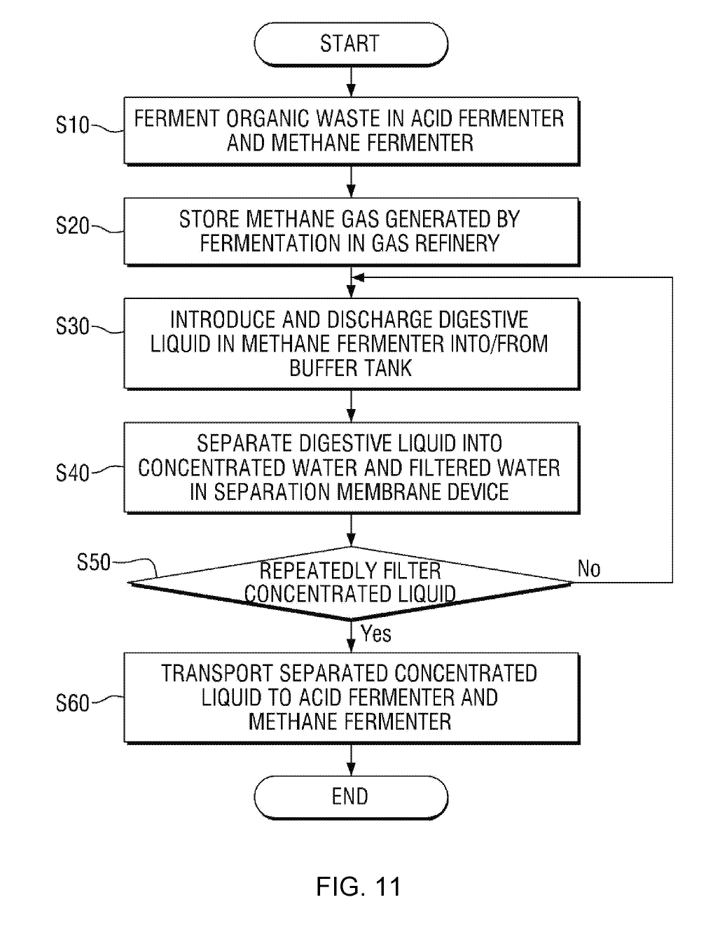

FIGS. 10A & 10B depict a method of controlling organic fouling and inorganic fouling that may be applied to the present disclosure.

The organic fouling control described in 10A & 10B may be performed one to four times in a day, and the inorganic fouling control may be performed one or two times in a month.

Referring to FIG. 10A, a method of adding about 0.1 mol of citric acid, about 0.9 mol of NaOH, and about 0.1 mol of NaOCl to basic water may be applied.

That is, as illustrated in FIG. 10B, water is inputted first for cleaning and then discharged, and water and NaOCl are inputted for cleaning and then discharged in order to control a phenomenon caused by deposition of the sludge or the like, and finally, only in a case in which inorganic substances are severely produced, the inorganic fouling control in which water and citric acid are inputted for cleaning and then discharged and then water and NaOH are inputted for cleaning may be performed.

<Method>

Hereinafter, an alternate operation control type membrane-coupled organic waste treatment apparatus and a method of operating the same according to the present disclosure will be described in detail with reference to the drawings.

FIG. 7 is a block diagram illustrating a organic waste treatment method using the alternate operation control type membrane-coupled organic waste treatment apparatus illustrated in FIG. 1.

First, the organic waste is supplied to the acid fermenter 140 and the methane fermenter 150 and fermented (S10). Of course, the organic waste is subjected to a pre-treatment process before the organic waste flows into the acid fermenter 140. The organic waste is hydrolyzed and acid-fermented while being stored in the acid fermenter 140 for approximately 3 to 4 days, such that high molecular organic compounds are converted into low molecular organic compound. In addition, a horizontal water flow is formed in the acid fermenter 140 by the first agitator 142, and a horizontal water flow and vertical circulation are performed at the lower side of the acid fermenter 140 by the agitation circulation line 143, such that the circulation is smoothly performed at the upper and lower sides of the acid fermenter 140. In this case, the agitation is performed in the acid fermenter 140 by the first agitator 142 and the turbulent flow formed by the agitation circulation line 143. Therefore, the agitation is performed at the upper side of the acid fermenter 140 by the first agitator 142, the water flow is formed at the lower side of the acid fermenter 140 by the agitation circulation line 143, and oil at the upper side of the acid fermenter 140 is smoothly mixed and agitated toward the lower side of the acid fermenter 140, thereby facilitating the acid fermentation. In addition, in the methane fermenter 150, a horizontal water flow is formed by the second agitator 154a, and upper scum is crushed and a vertical water flow is formed by the second agitator 154b, such that vertical and horizontal dual agitation is performed at the upper and lower side of the methane fermenter 150, thereby facilitating fermentation treatment.

In addition, the sludge, which flows horizontally and vertically, comes into contact with the inclined plate 260 and moves downward toward the bottom surface of the methane fermenter 150, such that the sludge is collected in the sludge groove 152. Of course, the sludge collected in the sludge groove 152 is discharged and removed to the outside at a predetermined interval. In addition, a part of the organic substance being fermented in the methane fermenter 150 is continuously circulated to the acid fermenter 140 through the first and second circulation lines 403 via the thickener tank 300 and the separation membrane device 400. In this case, the anaerobic digestive fluid in the methane fermenter 150, which is circulated to the acid fermenter 140, is supplied by about 10 to 20% of the amount of the anaerobic digestive fluid which is supplied from the acid fermenter 140 to the methane fermenter 150, such that pH 4.3 to 4.5 is continuously maintained with respect to the organic waste in the acid fermenter 140, thereby facilitating an acid fermentation reaction. Furthermore, even in the acid fermenter 140, the decomposition of the organic substance is performed by the anaerobic microorganism by about 5 to 10% of the acid fermentation decomposition amount. Of course, acidity may be increased to pH 5 to 6 by increasing the amount of organic substances to be supplied to the methane fermenter 150 in accordance with a degree of acid fermentation and an environment. Here, the organic substances are acid-fermented while being stored in the acid fermenter 140 for approximately 3 to 4 days, and the organic substance produces the digestive fluid and methane gas while being stored in the methane fermenter 150 for approximately 15 to 30 days.

Next, the biogas produced in the acid fermenter 140 and the methane fermenter 150 is stored in the gas storage tank 170 (S20). A part of the biogas in the gas storage tank 170 is provided to the boiler 180 which provides heat to the heat exchangers 160 connected to the acid fermenter 140 and the methane fermenter 150. Further, the remaining gas is provided to an electric power system or a high-purity gas refinery.

Next, the digestive fluid discharged from the methane fermenter 150 flows into the thickener tank 300 (S30). The digestive fluid, which is obtained by the reaction with the low molecular organic compound in the methane fermenter 150, flows into the thickener tank 300 through the supply line 301 and is agitated to prevent deposition.

A negative pressure is formed in the thickener tank 300 when the digestive fluid in the thickener tank 300 is discharged by the supply pump 320, and the negative pressure is compensated by the biogas generated in the thickener tank 300.

Next, the digestive fluid discharged from the thickener tank 300 is conveyed to the separation membrane device 400, and solid-liquid separated into a concentrated liquid and filtered water (S40). Specifically, the digestive fluid flowing into the thickener tank 300 is pressurized and conveyed to the separation membrane device 400 in a forward direction through the first digestive fluid inflow line 401. In this case, the first injection valve 401a and the first discharge valve 403a are in the opened state, and the second injection valve 402a and the second discharge valve 404a are in the closed state. Meanwhile, the digestive fluid is distributed and injected into the plurality of membrane filtration modules 420 via the first header 432. Further, as illustrated in FIG. 6, the water contained in the digestive fluid produces filtered water while penetrating the tubular filtration membrane 424, and thus the concentrated liquid with high concentration including the digestive microorganisms is discharged at a side opposite to the injection side. Further, the digestive fluid discharged from the plurality of membrane filtration modules 420 is collected in the second header 434 and discharged to the first concentrated liquid transport line 403. In this solid-liquid separation step S40, the injection direction of the digestive fluid is changed every predetermined time, thereby improving a lifespan of the membrane filtration module 420. Specifically, the injection direction of the digestive fluid is reversed as the control unit closes the first injection valve 401a and the first discharge valve 403a, and opens the second injection valve 402a and the second discharge valve 404a. Therefore, the digestive fluid flows into the tubular filtration membrane 424 through the second digestive fluid inflow line 402 and the second header 434, and the concentrated liquid is discharged to the first concentrated liquid transport line 403 through the first header 432 and the second concentrated liquid transport line 404 That is, an effect of removing contaminants stacked on the filtration membrane 424 to a certain degree is obtained by a change of the injection side of the tubular filtration membrane 424 and by the injection pressure in the opposite direction. In addition, in comparison with the related art in which membrane occlusion rapidly occur only at one end portion of the filtration membrane of the separation membrane device, contaminants are uniformly stacked on the entire membrane surface 424a of the tubular filtration membrane 424 of the present disclosure, thereby obtaining an effect of delaying occlusion of the membrane. This means an increase in lifespan of the tubular filtration membrane 424, and it is possible to reduce costs incurred due to frequent replacement of the filtration membrane 424. Meanwhile, a cycle on which the injection direction of the digestive fluid is reversed by the control unit may be approximately selected in a range from about 30 to about 60.

Next, the concentrated liquid, which is transported to the thickener tank 300 after the filtration in the separation membrane device 400, is discharged through the first concentrated liquid transport line 403, and in this case, whether the concentrated liquid is initially filtered or repeatedly filtered is determined (S40). That is, whether the methane fermented liquid is a low concentrated liquid obtained by being initially filtered and whether the initially filtered concentrated liquid is a high concentrated liquid which is filtered again by flowing back to the separation membrane device 400 from the thickener tank 300 are determined. In this case, the determination on the filtered state of the concentrated liquid may be performed based on predetermined concentration of the concentrated liquid in the thickener tank 300, and may be performed based on the number of times the concentrated liquid discharged from the separation membrane device 400 is filtered again. In the case in which the determination on the filtered state of the concentrated liquid is performed based on the concentration of the concentrated liquid, a concentration measuring unit (not illustrated) is further installed, and the filtration is repeatedly performed until the concentration becomes predetermined concentration or higher. In the case in which the determination on the filtered state of the concentrated liquid is determined based on the number of times the concentrated liquid is filtered, the determination is performed based on the number of times the initially filtered concentrated liquid is transported and filtered, and generally based on the case in which the number of times the concentrated liquid is filtered again is one. In addition, the determination may be performed based on the number of times the supply valve 320 installed in the supply line 301 between the thickener tank 300 and the separation membrane device 400 is opened and closed. That is, since a total amount of the methane fermented liquid or the concentrated liquid accommodated in the thickener tank 300 is supplied to the separation membrane device 400, the number of times the supply valve 320 is opened and closed is equal to a value made by adding the number of times the initially filtered concentrated liquid is supplied back to the separation membrane device 400 to one that is the number of times the methane fermented liquid initially flows in, and as a result, the number of times the supply valve 320 is opened and closed may be used as determination reference. Here, if the concentrated liquid is not in the repeatedly filtered state, the process goes to step S30 in which the methane fermented liquid in the methane fermenter 150 flows into the thickener tank 300. In this case, the high concentrated liquid flows to the transport line 302 of the methane fermenter 150 from the thickener tank 300 through the first concentrated liquid transport line 403 of the separation membrane device 400. Here, the circulation valve 302a installed in the first concentrated liquid transport line 302 is opened, and the second supply valve 403f for supplying the concentrated liquid to the first concentrated liquid transport line 403 and the acid fermenter 140 is closed. Next, in a state in which the concentrated liquid is repeatedly filtered to a desired degree, the concentrated liquid discharged from the second header 434 is transported to the acid fermenter 140 or the methane fermenter 150 through the first concentrated liquid transport line 403 (S40). Here, in the case in which the injection direction of the digestive fluid is changed as described above, the concentrated liquid is transported to the acid fermenter 140 or the thickener tank 300 through the second concentrated liquid transport line 404 and the first concentrated liquid transport line 403. In this case, the second supply valve 403f of the first concentrated liquid transport line 403 is opened, such that the repeatedly filtered high concentrated liquid flows into the acid fermenter 140 and the thickener tank 300. Here, the amount of the concentrated liquid flowing into the acid fermenter 140 and the thickener tank 300 is adjusted by the electronic flowmeter, the auxiliary valve, and the like. In addition, as necessary, the shut-off valve 301a is opened, such that the methane fermented liquid from the methane fermenter 150 is accommodated in the thickener tank 300.

Meanwhile, according to the present disclosure, a cleaning step of performing backwashing on the tubular filtration membrane 424 of the separation membrane device 400 may be further included. In the solid-liquid separation step S30, the first flowmeter 401b provided in the first digestive fluid inflow line 401 and the second flowmeter 403b provided in the first concentrated liquid transport line 403 measure a flow rate. However, in the cleaning step, the control unit calculates a difference between an injection flow rate of the digestive fluid and a discharge flow rate of the concentrated liquid based on a measured value, and when the derived difference in flow rate is smaller than a predetermined reference value, the solid-liquid separation step S300 and the concentrated liquid transport step S400 are stopped, and the backwashing is performed on the separation membrane device 400. In more detail, contaminants are stacked on the membrane surface 424a of the tubular filtration membrane 424 as time passed even though the injection direction of the separation membrane device 400 is changed, such that the amount of filtered water is decreased, and for this reason, the difference between the injection flow rate of the digestive fluid and the discharge flow rate of the concentrated liquid is gradually decreased. Therefore, the backwashing is performed when the life of the separation membrane device 400 is ended (performance deteriorates to a predetermined limitation). In this case, the reference value, which is to be compared with a difference in flow rate derived by the control unit, may be set based on data measured in a laboratory, and may be appropriately adjusted while the apparatus actually operates. For example, the reference value may be set based on a case in which the digestive fluid of about 5 tons is injected and the filtered water of 0.05 ton and the concentrated liquid of 4.45 tons are discharged. In addition, on the aforementioned principle, a difference in pressure is derived from the first manometer 401c and the second manometer 403c which are provided in the first digestive fluid inflow line 401 and the first concentrated liquid discharge line 403, respectively, and the backwashing may be performed on the separation membrane device 400 when the difference in pressure is smaller than a predetermined reference value.

The cleaning step is performed by the following processes.

First, the digestive fluid remaining in the tubular filtration membrane 424 of the separation membrane device 400 is discharged to the cleaning water supply line 601 by operating the supply pump 320. In this case, the digestive fluid is transported to the acid fermenter 140 and the methane fermenter 150 through the second concentrated liquid transport line 404 and the first concentrated liquid transport line 403.

Next, the backwashing pump 620 is operated, and the cleaning water stored in the water tank 610 is pressurized and injected into the tubular filtration membrane 424, thereby performing water cleaning for a predetermined time. In this case, the produced cleaning solution flows into the first concentrated liquid transport line 403 through the second concentrated liquid transport line 404, flows into the first cleaning solution transport line 405 by the closed fifth valve 403d, and then is transported to the raw water storage tank 412. In this case, the cleaning solution flows into the second cleaning solution transport line 406 and is stored in the linked storage tank 500, and the cleaning solution may then be sent to the wastewater treatment facility. Of course, the cleaning solution flows to the first cleaning solution inflow line 401, the first concentrated liquid transport line 403, and the first cleaning solution transport line 405, such that various flow directions of the cleaning solution may be implemented.

Next, a chemical is added into the cleaning water and the chemical cleaning is performed when the amount of permeable water is decreased on a cleaning cycle in the water cleaning step and an increase in pressure is smaller than a predetermined reference value. In more detail, because the cleaning effect is not perfect even though the water cleaning is performed on the separation membrane device 400, the cleaning cycle is inevitably decreased as time passed. For example, in a case in which the cleaning period is decreased from once in a week to once in three days, a chemical is added to the cleaning water, and the chemical cleaning is performed, and as a result, it is possible to further improve the lifespan of the tubular filtration membrane 424. The reason why the chemical cleaning is not performed from the first time is that in a case in which the chemical cleaning is often performed, fine pores in the tubular filtration membrane 424 are rapidly damaged and a large amount of costs is required to purchase the chemicals. Further, the cleaning solution produced by the chemical cleaning is transported and treated to the chemical tank 630 through the second concentrated liquid transport line L34 and the first cleaning solution transport line L50.

In steps S50 and S60, in the separation membrane device 400, the concentrated circulating water, which contains a large amount of anaerobic digestive sludge, is transported to the thickener tank 300 instead of being discharged to the outside.

In the case in which the concentrated circulating water containing a large amount of anaerobic digestive sludge is transported to the thickener tank 300 as described above, a level of the thickener tank 300 may be decreased by 50% or lower in comparison with the existing configuration.

Furthermore, in the present disclosure, the anaerobic digestive sludge contained in the concentrated circulating water is transported to the methane fermenter 150 once more, and as a result, it is possible to induce an effect of increasing the solid retention time (SRT) of the anaerobic microorganisms in the methane fermenter 150 and facilitating the decomposition of organic substances, and it is possible to prevent offensive odor from being produced.

In the case in which the anaerobic digestive sludge contained in the concentrated circulating water is transported to the methane fermenter 150 once more, it is possible to increase the amount of generated biogas by 20% or more and decrease the amount of sludge to be wasted by 30% or more in comparison with the existing method.

From the aforementioned description, it may be understood by a person skilled in the art that the present disclosure may be carried out in other specific forms without changing the technical spirit or the essential characteristics. Therefore, it should be understood that the above-described exemplary embodiments are illustrative in all aspects and do not limit the present disclosure. The scope of the present disclosure is represented by the claims to be described below rather than the detailed description, and it should be interpreted that the meaning and scope of the claims and all the changes or modified forms derived from the equivalent concepts thereto fall within the scope of the present disclosure.

What is claimed is:

1. An alternate operation control type membrane-coupled organic waste treatment apparatus which includes an acid fermenter, a methane fermenter, a thickener tank, and a separation membrane device, the alternate operation control type membrane-coupled organic waste treatment apparatus comprising:
a first circulation line in which a first circulation pump is installed in a linked manner so that a part of organic waste being acid-fermented in the acid fermenter is supplied to the methane fermenter; and
a second circulation line in which a second circulation pump is installed in a linked manner so that a part of a anaerobic digestive fluid being methane-fermented in the methane fermenter is supplied to the acid fermenter,
wherein the thickener tank is installed between the methane fermenter and the separation membrane device, supplied with concentrated circulating water from the separation membrane device, and supplies at least a part of the supplied concentrated circulating water to the methane fermenter.

2. The alternate operation control type membrane-coupled organic waste treatment apparatus according to claim 1, wherein the separation membrane device is formed in the form of a membrane having a pipe shape, and the membrane having the pipe shape is disposed to be twisted in at least one of horizontal and vertical directions.

3. The alternate operation control type membrane-coupled organic waste treatment apparatus according to claim 1, wherein the fluid flowing into the separation membrane device is moved in a first direction for a preset first period to supply the concentrated circulating water to the thickener tank, and when the first period is passed, the fluid is moved in a second direction opposite to the first direction to supply the concentrated circulating water to the thickener tank.

4. The alternate operation control type membrane-coupled organic waste treatment apparatus according to claim 1, wherein the separation membrane device periodically controls organic fouling and inorganic fouling by using a fluid including at least one of water, citric acid, NaOH, and NaOCl,
wherein the organic waste treatment apparatus further includes:
a separator and crusher which separates and crushes the organic waste;
a pulverizer which pulverizes the organic waste passing through the separator and crusher to a size suitable to be inputted into the acid fermenter;
a screen which filters the organic waste having a predetermined size or larger from the organic waste passing through the pulverizer;
a storage tank which temporarily stores the organic waste passing through the screen;
heaters which are installed in the acid fermenter and the methane fermenter, respectively, in order to increase a temperature of the organic waste in the acid fermenter and the methane fermenter to a predetermined temperature;
a gas storage tank which stores methane gas generated in the methane fermenter; and
a boiler into which the methane gas in the gas storage tank flows and which provides heat to the heaters.

5. The alternate operation control type membrane-coupled organic waste treatment apparatus according to claim 1, wherein the methane fermenter includes:
a plurality of sludge grooves which is formed at a rim portion of a bottom surface of the methane fermenter so as to collect sludge that comes into contact with an inclined plate and moves downward; and
a sludge discharge line which is connected to the sludge grooves and discharges the sludge.

6. The alternate operation control type membrane-coupled organic waste treatment apparatus according to claim 1, wherein a bottom surface of the methane fermenter is inclined downward from a center to a rim.

7. The alternate operation control type membrane-coupled organic waste treatment apparatus according to claim 1, wherein the methane fermenter includes a second agitator installed such that a second agitation blade is installed in the methane fermenter so as to be rotated by a second agitation motor.

8. The alternate operation control type membrane-coupled organic waste treatment apparatus according to claim 1, wherein the acid fermenter includes a first agitator installed such that a first agitation blade is installed in the acid fermenter so as to be rotated by a first agitation motor.

9. The alternate operation control type membrane-coupled organic waste treatment apparatus according to claim 1, wherein the acid fermenter further includes an agitation circulation line which is installed to connect upper and lower sides of the acid fermenter and in which an agitation circulation pump for supplying the organic waste from the upper side to the lower side is installed in a linked manner.

10. The alternate operation control type membrane-coupled organic waste treatment apparatus according to claim 1, wherein the second circulation line is connected to the agitation circulation line from the methane fermenter.

11. The alternate operation control type membrane-coupled organic waste treatment apparatus according to claim 1, wherein the thickener tank includes a gas holder installed at an upper side of the thickener tank in order to compensate for a negative pressure generated when the digestive fluid accommodated in the thickener tank is discharged,
wherein the gas holder is made of a soft material and installed to be contracted while discharging collected gas when the negative pressure is generated in the thickener tank.

12. The alternate operation control type membrane-coupled organic waste treatment apparatus according to claim 1, further comprising:
a second digestive fluid inflow line which diverges from a first digestive fluid inflow line connected to the thickener tank and is connected to multiple stages of the separation membrane device;
a second concentrated liquid transport line which is connected to the separation membrane device and connects to the first concentrated liquid transport line installed to supply the concentrated liquid to the acid fermenter and the methane fermenter;
a first injection valve which is provided in the first digestive fluid inflow line;
a first discharge valve which is provided in the first concentrated liquid transport line;
a second injection valve which is provided in the second digestive fluid inflow line;
a second discharge valve which is provided in the second concentrated liquid transport line; and
an injection direction changing means which includes a supply pump installed in the first digestive fluid inflow line, and a control unit for controlling the first and second injection valves and the first and second discharge valves,
wherein the injection direction changing means reverse the injection direction of the digestive fluid into the separation membrane device every predetermined time,
wherein the first concentrated liquid transport line is connected to the supply line so that the concentrated liquid circulates to the thickener tank.

13. The alternate operation control type membrane-coupled organic waste treatment apparatus according to claim 12, further comprising:
a circulation valve which is installed in the first concentrated liquid transport line so that the concentrated liquid flowing through the first concentrated liquid transport line flows into the supply line or is blocked;
a first supply valve which is installed so that the concentrated liquid flowing through the first concentrated liquid transport line is supplied to the acid fermenter or blocked;
a second supply valve which is installed so that the concentrated liquid flowing through the first concentrated liquid transport line is supplied to the methane fermenter or blocked; and
a shut-off valve which is opened and closed so as to block a flow of the digestive fluid discharged from the methane fermenter.

14. The alternate operation control type membrane-coupled organic waste treatment apparatus according to claim 13, wherein the shut-off valve, the first supply valve, and the second supply valve are closed when the circulation valve is opened, and the first supply valve and the second supply valve are opened when the circulation valve is closed,
wherein the shut-off valve is installed to be opened only when the digestive fluid is supplied to the thickener tank.

15. A method of treating organic waste, the method comprising:
a first step S10 of supplying organic waste in an acid fermenter and a methane fermenter and fermenting the organic waste;
a second step S20 of storing methane gas generated in the first step S10 in a gas storage tank;
a third step S30 of introducing and discharging a digestive fluid discharged from the methane fermenter or a concentrated liquid discharged from the a separation membrane device to/from a thickener tank through a supply line;
a fourth step S40 of injecting, by a supply pump, the digestive fluid stored in the thickener tank into the separation membrane device to separate the digestive fluid into a concentrated liquid and filtered water, and reversing an injection direction of the digestive fluid every predetermined time;
a sixth step S60 of transporting the separated concentrated liquid to the acid fermenter and the methane fermenter;
a seventh step of allowing the thickener tank to be supplied with concentrated circulating water from the separation membrane device; and
an eighth step of supplying at least a part of the concentrated circulating water supplied to the thickener tank to the methane fermenter.
